(19) United States
(12) Patent Application Publication
(10) Pub. No.: US 2008/0135441 A1
(43) Pub. Date: Jun. 12, 2008

(54) Blister pack

(75) Inventors: Andreas Mark Meliniotis, Cambridge (GB); Peter Alan Evans, Cambridgeshire (GB); Stephen William Eason, Norfolk (GB); Quentin John Harmer, Cambridge (GB)

Correspondence Address: Davidson, Davidson & Kappel, LLC, 485 7th Avenue, 14th Floor, New York, NY, 10018, US

(73) Assignee: Vectura Group PLC, Chippenham (GB)

(21) Appl. No.: 11/919,779

(22) PCT Filed: Apr. 13, 2006
(86) PCT No.: PCT/EP2006/061606

§ 371 Date: Dec. 26, 2007

(30) Foreign Application Priority Data
Apr. 15, 2005 (GB) 0507710.2

(51) Int. Cl. B65D 83/04; B65B 61/18; B65B 61/20; B65B 7/00; A61M 15/00

(52) U.S. Cl. 206/531; 53/412; 53/133.8; 53/285; 128/203.12

(57) ABSTRACT

A blister pack comprising a plurality of spaced blister cavities each configured to receive and store an individual dose of medicament for inhalation by a user is disclosed. The pack includes a foil layer and an outer polymer layer and each blister cavity, or a number of blister cavities, are separated from an adjacent blister cavity, or number of adjacent blister cavities, by a region of weakness formed by substantially removing or displacing a portion of the outer polymer layer from the foil layer.

### DESCRIPTION

The present invention relates to a blister pack and, in particular, to a strip of blisters that are used to store individual doses of medicament in dry powdered form prior to the sequential inhalation of each dose by a patient using an inhalation device equipped with an indexing and piercing mechanism and in which the strip is pre-loaded or fitted by the patient ready for use. The invention also relates to a method of imparting a line of weakness in a blister pack according to the invention, a device or sealing tool for imparting said line of weakness either during or after manufacture of the blister pack and, an inhalation device containing a strip of blisters according to the invention.

Oral or nasal delivery of a medicament using an inhalation device is a particularly attractive method of drug administration as these devices are relatively easy for a patient to use discreetly and in public. As well as delivering medicament to treat local diseases of the airway and other respiratory problems, they have more recently also been used to deliver drugs to the bloodstream via the lungs thereby avoiding the need for hypodermic injections.

In one type of conventional metered dose inhalation device, the powdered medicament is held in a reservoir within a dispensing device that is operable to measure out and dispense a predetermined amount of powder for each dose. However, these devices suffer from poor dose metering capability especially when the size of the dose is relatively small as it is difficult to accurately measure out small amounts of dry powder in such a device. It is also difficult to protect the drug from the ingress of moisture and to seal it from the atmosphere until it is required for administration to a patient.

In view of the foregoing, it has become common for dry powder formulations to be pre-packaged in individual doses, usually in the form of capsules or blisters that each contain a single dose of the powder which has been accurately and consistently measured. A foil blister is preferred over capsules as each dose is protected from the ingress of water and penetration of gases such as oxygen in addition to being shielded from light and UV radiation all of which can have a detrimental effect on the delivery characteristics of the inhaler if a dose becomes exposed to them.

Inhalation devices that receive a blister pack or strip of blisters are known. Actuation of the device causes a mechanism to index and pierce a blister so that when the patient inhales, air is drawn through the blister entraining the dose, which is then carried out of the blister through the device and via the patient's airway down into the lungs.

A blister pack generally comprises a base having a number of spaced apart cavities defining blisters to receive individual doses of medicament and, a lid in the form of a generally planar sheet that is sealed to the base except in the region of the cavities using a sealing tool which compresses the base and lid material together in a region surrounding each cavity. The tool is heated so that the lid is sealed to the base during the compression step. The base material is typically a laminate comprising a polymer layer in contact with the drug, a soft tempered aluminium layer and an external polymer layer. The aluminium provides the moisture and oxygen barrier, whilst the polymer aids the adherence of the foil to the heat seal lacquer and provides a relatively inert layer in contact with the drug. Soft tempered aluminium is ductile so that it can be “cold formed” into a blister shape. It is typically 45 μm thick. The outer polymer layer provides additional strength and toughness to the laminate.

The lid material is typically a laminate comprising a heat seal lacquer, a hard rolled aluminium layer (typically 20-30 μm thick) and an external lacquer layer. The heat seal lacquer bonds to the polymer layer of the base foil laminate during heat-sealing to provide a seal around the top of the blister cavity. The aluminium layer is hard rolled to facilitate piercing of the blister by the inhalation device when access to the medicament contained therein is required. Materials for the polymer layer in contact with the drug include poly vinyl chloride (PVC), polypropylene (PP) and polyethylene (PE). In the case of PE, the heat seal lacquer on the foil lid is replaced with a further layer of PE. On heat sealing, the two layers of PE melt and weld to each other. The external polymer layer on the base foil is typically oriented polyamide (oPA). The polymer layer in contact with the drug is typically PVC of 60 μm thickness. However, a thinner layer of 30 μm or 15 μm may be used, for example, where a more flexible laminate is required.

It will be appreciated that different types of medicament possess varying degrees of sensitivity to various environmental influences and so a foil blister of the type described above provides good environmental protection for the medicament and protects it against moisture ingress, oxygen and other gases. The foil conveniently also protects the drug from light. Although the foil material itself is impermeable to moisture and gases, providing it is not punctured, the polymer layers are permeable to a greater or lesser extent. The permeability is typically defined by a moisture or gas transmission rate over a given time. The transmission rate depends on the type of material, the thickness of the permeable layer and distance of the transmission path. Thus the level of protection provided is determined in part by the breadth of the seal around the blister as this determines the distance any moisture or oxygen has to travel through the polymer layer from the edge of the foil laminate to the blister cavity.

In a strip of blisters, ingress can occur from the edges of the strip or from an adjacent blister that has been punctured. Thus the required breadth of seal should be maintained both from the blister cavity to the edges of the strip and from one blister cavity to an adjacent blister cavity. This distance between the blister cavities or seal breadth should be at least 2 mm although at least 2.5 mm is more preferable when the medicament is not particularly sensitive to environmental factors. However, a greater distance such as 3, 4 or 5 mm or more will afford improved environmental protection and should be used when the medicament is more sensitive to environmental factors.

It is desirable for an inhalation device, such as those used to treat a respiratory disease such as asthma or COPD, to be able to contain sufficient doses for at least one month's treatment. Typically, this requires an inhaler with 30 blisters (for a once daily dose) or 60 blisters (for a twice daily dose). It is known from GB2242134 to provide a device that is capable of receiving an individually sealed foil blister strip of 60 doses in which the lid is peeled away from the base of the strip by the device to enable access to the dose to be obtained. However, the device disclosed in this document is provided with chambers to receive both the used blister base and the lid that has been peeled away from the base and this makes the device unnecessarily large.

An alternative approach is to facilitate the detachment of used blisters from the unused blisters that remain in the strip so that the used blisters may be discarded. This allows the device to be smaller as there is no longer any requirement to store used blisters in the device.

A problem with detaching used blisters is that the external and internal polymer layers on the base foil laminate make it tough and difficult to tear. It is therefore known, for example from EP0469814A, to provide the strip with a series of perforations in the foil between blisters to facilitate their separation by tearing along the perforations. However, when a strip is provided with perforations, the distance between blisters has to be increased and maybe even doubled because the foil is cut by the perforating process thereby creating a break in the moisture seal. Increasing the distance between adjacent blisters increases the sealing distance, i.e. the distance moisture or gas has to travel to reach the drug, and so restores the environmental protection to a similar level found in a blister strip that is not provided with perforations. However, a disadvantage with increasing the distance between adjacent blisters is that the resulting blister strip is considerably longer and so a larger device is required to contain them. Furthermore, in a device that is equipped with an indexing mechanism for incrementally advancing the blisters to a piercing position, an increase in the distance between blisters requires a greater incremental movement to advance the blister strip leading to an increase in the complexity or size of the indexing mechanism.

The present invention seeks to provide a blister pack which is tearable but overcomes or substantially alleviates the problems associated with a perforated strip. In particular the invention seeks to provide a strip which facilitates easy separation of used blisters from those that remain and enables the minimum distance between blisters to be maintained without compromising the integrity of the seal between the blisters and the environmental protection provided by the seal.

According to the present invention, there is provided a blister pack comprising a plurality of spaced blister cavities each configured to receive and store an individual dose of medicament for inhalation by a user, wherein the pack includes a foil layer, an outer polymer layer and a region of weakness formed by substantially removing or displacing a portion of the outer polymer layer from the foil layer between each blister cavity, or a number of blister cavities.

In one embodiment, the package includes an inner polymer layer on the foil and the region of weakness is formed by substantially displacing the inner polymer layer, in addition to substantially removing or displacing the outer polymer layer, from the foil.

The blister pack preferably comprises a base portion in which the blister cavities are formed and a substantially planar lid portion sealing the blister cavities.

Preferably, the blister package is in the form of an elongate strip of blisters and, most preferably, a region of weakness is provided between each blister of the strip. Advantageously, each region of weakness is in the form of a straight narrow strip or line and extends across the strip substantially at right angles with respect to the longitudinal edges of the strip.

The strip is preferably sufficiently flexible to enable it to be wound into a roll for insertion into an inhalation device equipped with an indexing mechanism for advancing the blisters one at a time to a piercing station to enable the dose contained therein to be accessed and inhaled by a patient.

Although the region of weakness may be unbroken, it is also envisaged that, in one embodiment, one or more regions of weakness may be discontinuous. In this arrangement, the outer polymer layer, and possibly the inner polymer layer, are substantially removed or displaced from discrete, spaced apart regions extending along each region of weakness so that the region of weakness is formed from a series of weakened and non-weakened sections.

In a preferred embodiment, a region of weakness may be provided at an edge of a pack to facilitate the initiation of a tear As the force required to initiate a tear is greater than the force required to continue tearing once a tear has been initiated, a region of weakness at the edge of the pack is alone sufficient to enable separation.

In one embodiment, an edge of the pack may include a notch or a nick or a perforation or a region which has been highly compressed, scored or impacted or a region that has been heated and compressed or otherwise weakened to facilitate the initiation of a tear.

The blister package may comprise a lid and a base wherein the blisters are formed in the base, the region of weakness also being formed in the base.

In an alternative embodiment, regions of weakness are formed by locally melting or ablating or otherwise weakening the outer polymer layer. In a preferred embodiment a laser locally melts, ablates or softens or otherwise weakens the outer polymer layer.

In alternative embodiments regions of weakness may be formed by scoring with a rotary or straight blade (often called “kiss cutting”) or mechanically forming by nipping between two edges or local impact or by local pressure.

If the blister package is provided with means for initiating a tear where the regions of weakness meet the edge of the package to facilitate the start of a tear along the regions of weakness, these can be formed by any suitable means including kiss cutting, perforation, die cutting, application of a hot tool, application of pressure or laser ablation.

In a preferred embodiment, at least a portion of the region of weakness is a narrow strip or line. The region of weakness may also include an enlarged region where the line meets an edge of the pack. In one particular embodiment, the narrow strip or line extends across the pack between two enlarged regions where the ends of the line meet the edges of the pack.

According to the present invention, there is also provided a method of imparting a region of weakness in a blister pack between adjacent blister cavities of the pack which receive and store individual doses of medicament for inhalation by a user, the pack including a foil layer and an outer polymer layer, wherein the method includes the step of substantially removing or displacing a portion of the outer polymer layer from the foil layer to form said region of weakness.

The step of substantially removing or displacing a portion of the outer polymer layer from the foil layer to form said region of weakness preferably includes the step of applying heat and pressure to the pack to soften or melt a portion of the outer polymer layer and compress and/or push said portion away from the foil in said region.

In one embodiment, the method includes the step of cutting a portion of the pack in a region where a region of weakness meets an edge of the pack to form means for initiating a tear in the region of weakness.

According to the present invention, there is also provided a device for imparting a region of weakness to a blister pack between adjacent blister cavities of the pack which receive and store individual doses of medicament for inhalation by a user, the pack including a foil layer and an outer polymer layer, the device including means for heating and/or compressing a portion of the blister pack in a region where a region of weakness is to be formed so as to substantially remove or displace a portion of the outer polymer layer from the foil layer.

The device preferably comprises a heated blade member. The device may also include a base member on which the blister pack is located and stop members depending from the heated blade member that engage the base member during formation of a region of weakness to maintain a predetermined distance between the heated blade member and the base member.

The device may advantageously include cutting members depending from the heated blade member to cut a portion of the pack in a region where a region of weakness meets the edge of the pack to form means for initiating a tear in a line of weakness.

In another embodiment, the means for heating a portion of the blister pack in a region where a region of weakness is to be formed so as to substantially remove or displace a portion of the outer polymer layer from the foil layer is a laser which may be configured so as to remove or displace a portion of the outer polymer layer from discrete separate regions along each region of weakness.

The present invention also provides an inhalation device containing a blister pack according to the invention.

Although the blister package of the present invention is intended for use in many different devices, it is primarily intended for use in the inhalation device disclosed in the Applicant's co-pending international PCT application No. PCT/GB2004/004416 published as WO2005/037353 A1, which includes an actuator for indexing and piercing each of the blisters and in which used blisters protrude from the housing to facilitate their removal from those unused blisters that remain in the housing.

In the aforementioned device, a strip of blisters is coiled inside the device. However, it has been established that coiling the strip can cause the lidding foil to come under excessive stress in the vicinity of the regions of weakness. This is caused by the thickness of the laminate and the concentration of stress due to the thinning of the laminate at the region of weakness. These problems are mitigated by using a cold formed foil with a thinner polymer layer in contact with the drug. The polymer layer in contact with the drug is preferably less than 60 μm and, most preferably, between 15 μm and 40 μm thick. In a particularly preferred embodiment, the thickness of the polymer layer is 30 μm. Not only does a thinner polymer layer reduce the stress applied to the lidding foil when the strip is coiled, but a thinner polymer layer also makes the laminate much easier to tear, especially once a tear has been initiated.

According to another aspect of the invention, there is provided a sealing tool for manufacturing a blister pack, the pack comprising a base portion having a plurality of blister cavities therein and a lid portion for attachment to the base portion over the cavities to seal them once a dose has been located in each blister cavity, the pack including a foil layer and one or more polymer layers, wherein the sealing tool comprises a heated surface to compress and heat a blister base and lid positioned on the base to heat seal the lid to the base, wherein the sealing surface includes raised sections to substantially compress the laminate and remove or displace a portion of any outer polymer layer from the foil layer to form a region of weakness as the lid is heat sealed to the base.

In a preferred embodiment, the heated surface is knurled and the raised regions are substantially level with the topmost surfaces of the knurling pattern. Alternatively, the raised region is proud of the knurled pattern by up to 0.2 mm.

In one embodiment, the raised region has a first section to form a region of weakness at the edge of a pack and a second section to form another region of weakness extending from the first section remote from the edges of the pack. The height of the first section from the surface may be greater than the height of the second section from the surface.

Embodiments of the present invention will now be described, by way of example only, with reference to the accompanying drawings, in which:

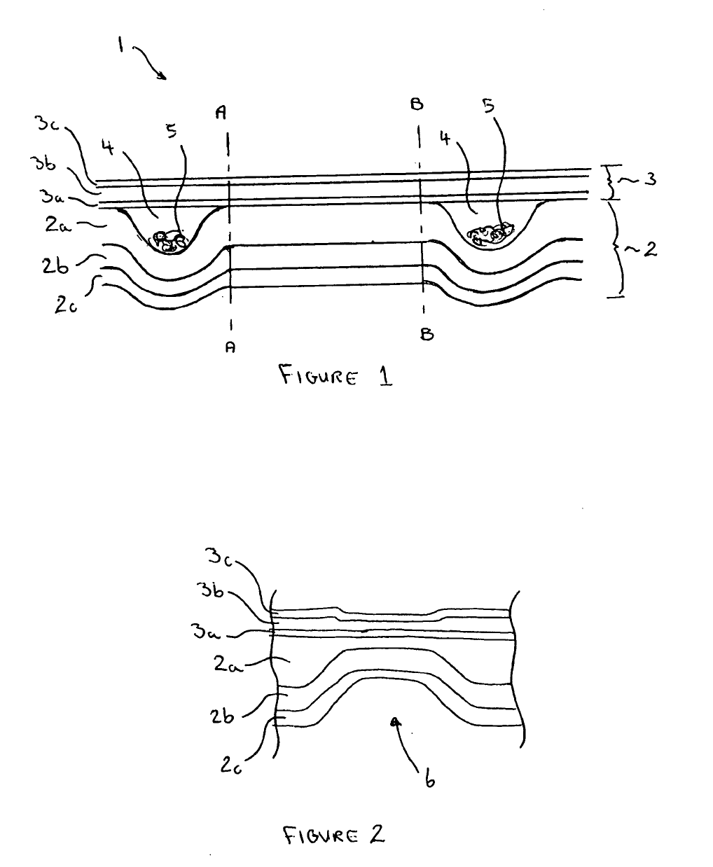

FIG. 1 illustrates a side-sectional view through a portion of a strip of blisters showing two adjacent blisters prior to processing of the strip to form a line of weakness between them, the thickness of the material layers from which the strip is formed being shown grossly over exaggerated to facilitate understanding of its construction;

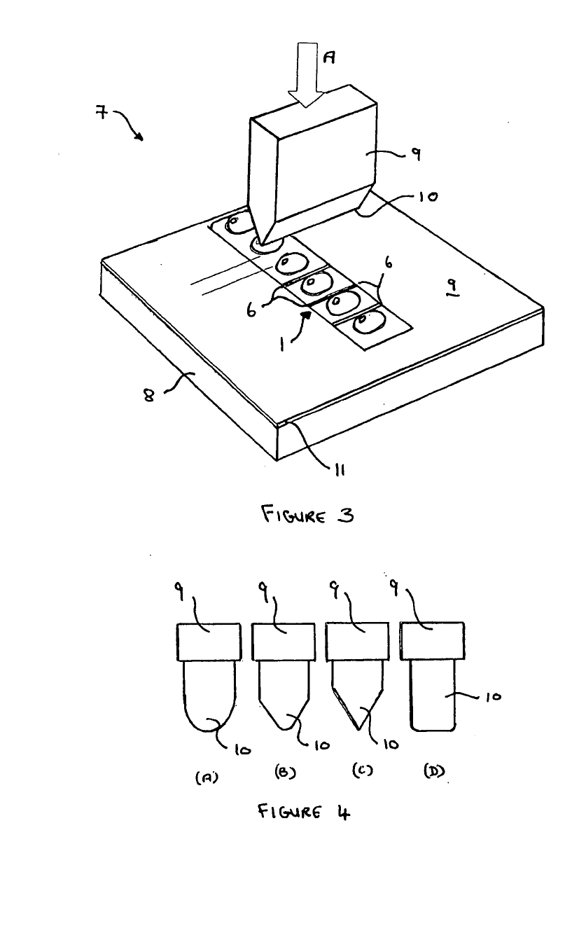

FIG. 2 illustrates a portion of the cross sectional view of FIG. 1 between the lines A-A and B-B shown in FIG. 1 and after a line of weakness between adjacent blisters has been formed;

FIG. 3 illustrates a simplified perspective view of a tool that is used to form the lines of weakness between blisters and shows a strip of blisters in which three lines of weakness have already been formed;

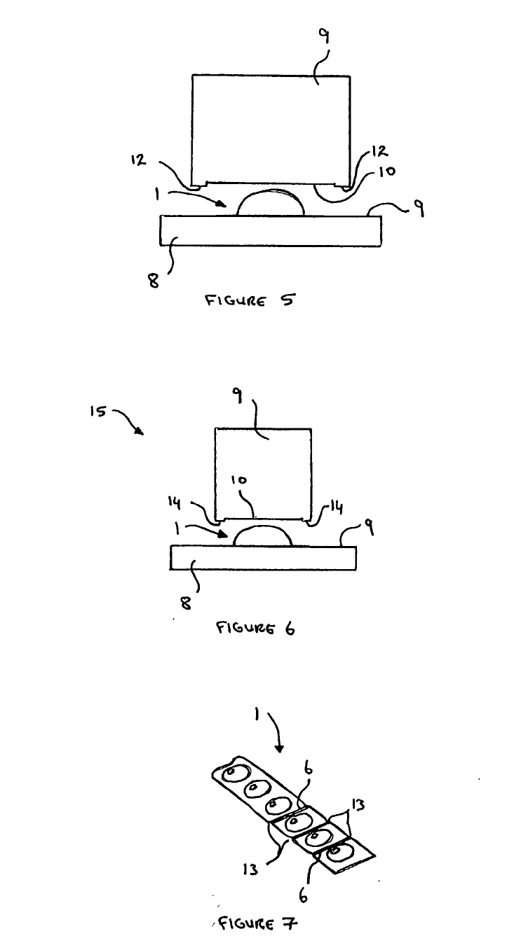

FIGS. 4A to 4D illustrate alternative cross-sections through the blade member of the tool shown in FIG. 3;

FIG. 5 illustrates a side sectional view of the blade member of the tool shown in FIG. 3 but in which the blade member is provided with stops to control the position of the blade;

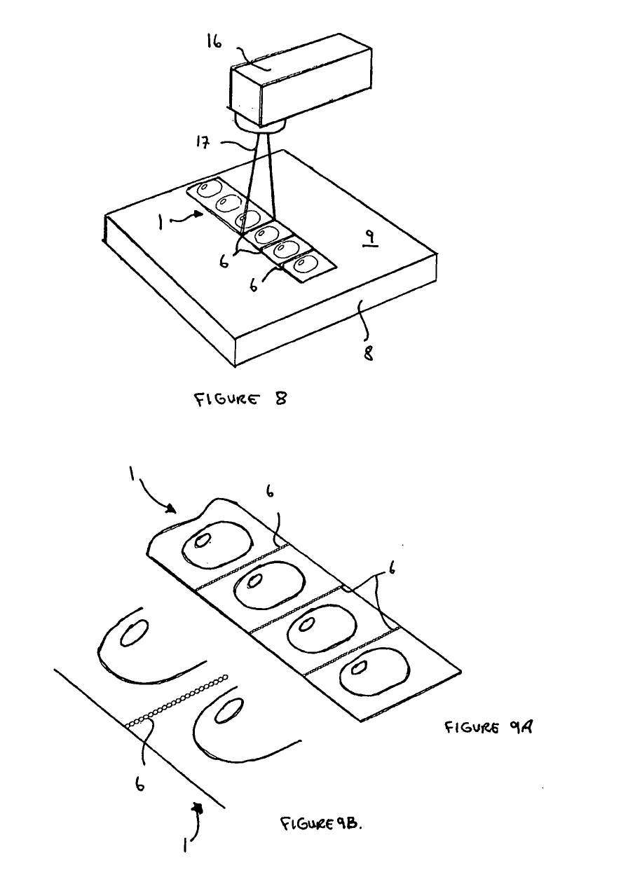

FIG. 6 illustrates a side sectional view of the blade member of the tool shown in FIG. 3 but in which the blade member is provided with cutting elements on each edge to cut a notch in the blister strip at either end of the line of weakness formed by the blade member;

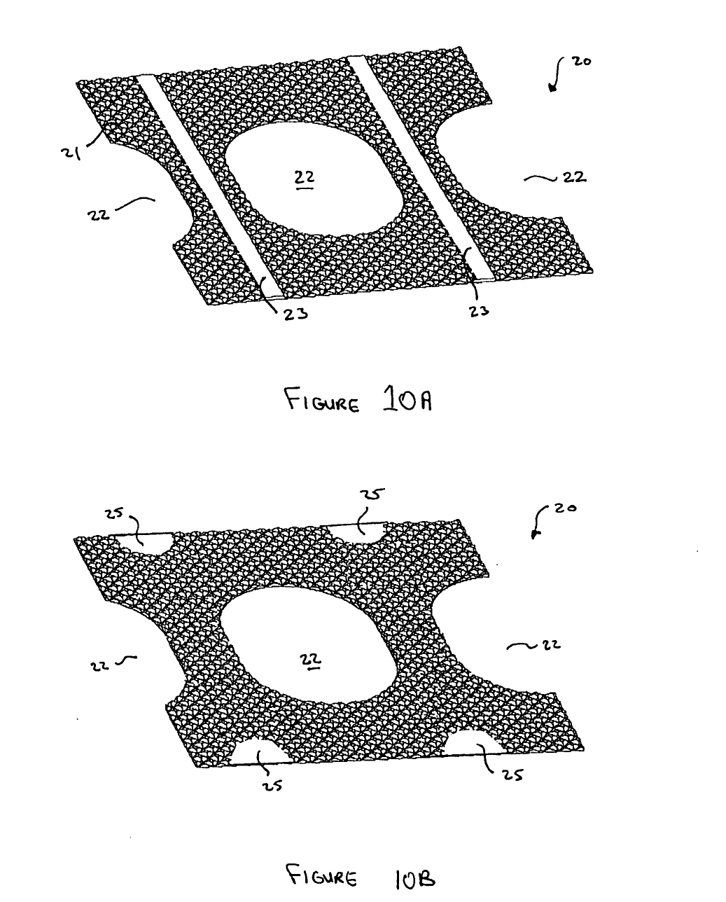

FIG. 7 illustrates a perspective view of a blister strip which has in which three lines of weakness have been formed between adjacent blisters and notches have been cut at each end of each line of weakness, using the blade member shown in FIG. 6;

FIG. 8 illustrates an alternative tool for forming the lines of weakness between blisters;

FIG. 9A and 9B illustrate a perspective view of a strip of blisters and, an enlarged perspective view of a line of weakness extending between adjacent blisters respectively, and which have been formed using the alternative tool illustrated in FIG. 8; and

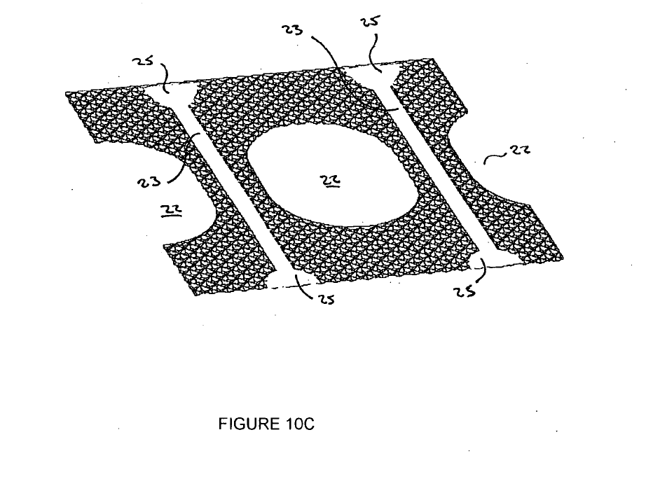

FIGS. 10A, 10B and 10C illustrate three different versions of a portion of a blister sealing tool which forms regions to facilitate the initiation and/or propagation of a tear between blister cavities.

Referring now to the drawings, there is shown in FIG. 1, a cross section through a portion of a strip of blisters 1 showing two blisters 4 and a portion of the strip that extends between them and in which a line of weakness 6 is to be formed. Although the invention is described with reference to a elongate strip 1 of blisters which are sufficiently flexible to enable them to be coiled for insertion into an inhalation device, such as the device disclosed in the Applicant's co-pending application No. 0324358.1, it will be appreciated that the blister pack 1 of the invention can take many different forms and configurations.

The blister strip 1 shown in FIG. 1 comprises a base portion 2 and a lid portion 3. The base portion 2, in which the blister cavities 4 each containing a dose of medicament 5 are formed, is a laminate of three layers: a polymer layer 2a which contacts the medicament, a soft tempered aluminium foil layer 2b and an external polymer layer 2c. The lid portion 3 is a planar laminate sheet formed from three layers: a heat seal lacquer layer 3a which is bonded to the polymer layer 2a of the base portion 2 during heat sealing to provide a seal around the top of the blister cavity 4, an aluminum foil layer 3b and an external lacquer layer 3c. The specific materials and constructional aspects of the blister pack 1 have already been described above and so will not be repeated again.

In a modified blister strip 1, the base portion 2 may include an additional polymer layer (not shown) on the side away from the blister cavity 4 to create a more symmetric laminate that is less susceptible to warping or distortion during the cold forming of the blister cavities 4.

FIG. 2 shows a portion of the blister strip 1 between the lines A-A and B-B shown in FIG. 1, after a line of weakness 6 has been formed therein between adjacent blister cavities 4. It will be appreciated that the thickness of the laminates in the region of the line of weakness 6 have been greatly reduced and compressed and that some of external polymer layer 2c of the base portion 2 has been displaced to reduce its thickness, although it is also envisaged that a portion of the external polymer layer 2 may be removed altogether. An important aspect of the invention is that the foil layer 2b,3b of the base portion 2 and of the lid portion 3 remains undamaged and unbroken despite the formation of the line of weakness 6.

As the force required to initiate a tear in a blister strip 1 at a line of weakness 6 is greater than the force required to continue tearing once a tear has been initiated, initiating features 13 may be provided at one or both of the edges of each line of weakness 6 to facilitate the initiation of a tear. The initiator 13 may be a notch, a nick or a perforation or a region which has been highly compressed, scored or impacted or a region that has been heated and compressed or otherwise weakened. A strip 1 of blisters in which each line of weakness 6 is provided with a tear initiator 13 is illustrated in FIG. 7. It will be appreciated that a initiator 13, such as a cut, at the edge of the strip 1 between the blisters 4 does not affect the minimum seal breadth from the edge of the strip 1 to the blister cavity 4 providing that the initiator 13 is outside a region defined by one cavity 4 to cavity 4 distance from the perimeter of the cavity 4.

Tools for, and methods of, forming the line of weakness 6 shown in the blister strip 1 of FIG. 2 will now be described with reference to FIGS. 3 to 9.

FIG. 3 shows a tool 7 comprising a table 8 having an upper surface 9 and a heated blade member 9 disposed above the table 8 which is provided with a mechanism (not shown) for reciprocating the blade member 9 towards and away from the upper surface of the table 8 in the direction indicated by arrow “A”. The heated blade member 9 includes a tool tip 10 for engagement with a blister strip 1 located on the upper surface of the table 8. A predetermined pressure is applied to the blister strip 1 by the blade member 9 when it moves towards and into contact with a blister strip located on the table 8 to compress the blister strip and the heat causes the external polymer layer 2c to melt and soften causing it to be partially or wholly displaced in a region where the blade member 9 contacts the blister strip 1. Although it is intended that the blister strip 1 is placed face-down on the table 8, i.e. with its lid portion 3 against the upper surface 9 of the table 8 so that the tool tip 10 engages and forms the line of weakness in the base portion 2, it will be appreciated that the blister strip 1 may also be placed the other way up on the table 8 so that the tool tip engages the lid portion 3 of the strip 1.

Suitable materials for the blade member 9 include aluminium and aluminium alloys, preferably hard anodised, and stainless steels. Advantageously the blade member 9 may be coated with a low friction or “non-stick” coating such as PTFE (polytetraflourethylene) to help ensure that the outer polymer layer 2c on the base portion 2 does not adhere to the blade member 9 during compression and heating, as material adhering to the blade member 9 would reduce the effectiveness of the line of weakness formation.

If the blister strip 1 is modified by the presence of an additional polymer layer (not shown), this additional layer can help reduce the propensity of the foil layer 2b to adhere to the blade member 9, especially if the additional polymer layer is formed from a material that is less susceptible to “stringiness” when it softens, for example PVC.

The tool tip 10 of the blade member may have a radius of 0.2 to 1.0 mm and more preferably 0.4 to 0.6 mm. In a particularly preferred embodiment, the radius of the tool tip 10 is 0.5 mm. A cross-section through the tool tip 10 is shown in FIG. 4A. However, it is also envisaged that the tool tip 10 may take alternative configurations to include a V-shaped blade with a radius at the tip (FIG. 4B), a V-shaped blade with no radius at the tip (FIG. 4C) or a flat blade (FIG. 4D).

The table 8 may be formed from aluminium and/or aluminium alloys, preferably hard anodised, stainless steels and high temperature polymers such as PEEK (poly ether ether ketone), polyamide or PTFE. Where required, for example in the case where the medicament 5 is sensitive to temperature, the table 8 may be cooled.

The upper surface 9 of the table 8 may optionally be provided with a thin resilient layer 11. Layer 11 assists in the formation of the line of weakness 6 by reducing the sensitivity of the process to the level of force applied and allows the foil layers 2b,3b of the blister strip 1 to bend slightly during forming so that the stresses, particularly shear and tearing stresses, induced in the foil layers 2b,3b are reduced thereby ensuring that they are not broken or cut by high levels of force. Suitable materials for the resilient layer 11 include polyamides, polyimides, PTFE, ETFE and silicone rubbers. The layer 11 is preferably less than 1 mm thick and more preferably less than 0.5 mm. In a preferred embodiment the resilient layer 11 is formed from a 0.3 mm layer of polyamide.

To ensure that the outer and inner polymer layers 2a,2c are softened sufficiently and so that the material of the outer polymer layer 2c is squeezed to the sides of the blade member 9 without cutting the foil layers 2b,3b, it is important to carefully select the temperature of the blade member 9 and the duration of contact with the blister pack 1. Suitable and preferred ranges for the key operating parameters are shown in the table below. It will be clear to those skilled in the art that the parameters interact with each other, for example increasing the duration will allow a lower force to be applied and increasing temperature may allow a shorter duration to be employed.

| | Suitable range | Preferred range |
|---|---|---|
| Top tool temperature (° C.) | 230-280 | 235-245 |
| Force per tear line (N) | 300-600 | 400-500 |
| Duration (s) | 0.1-3.0 | 0.5-1.0 |

The application of heat and pressure needs to be controlled to achieve a repeatable line of weakness 6. One option is to control the force or pressure applied by, for example, using a spring at a predefined level of compression or a pneumatic cylinder at a predetermined pressure to provide the force. Alternatively, the blade member 9 may be provided with one or more stop members 12 which holds the tool tip 10 a predetermined distance from the upper surface 9 of the table 8 so that the blister strip 1 is compressed only by a predetermined amount by the blade member 9. In the embodiment illustrated in FIG. 5, stop members 12 are provided on each edge of the tool tip 10. The distance between the tool tip 10 and the upper surface 9 of the table 8 may be adjustable to enable lines of weakness 6 to be formed in blister strips 1 having various thicknesses of laminate. Preferably the distance between the tool tip 10 and the upper surface 9 is selected to be between 25% and 100% of the total thickness of the laminate.

If a tear initiator 13 is to be provided, this can be formed in the same operation as the formation of the line of weakness 6. FIG. 6 shows an alternative tool 15 for forming a line of weakness 6 together with notches at each end of the line of weakness 6 to facilitate the initiation of a tear. Cutting elements 14 depend from the heated tool member 9 and are received in mating recesses (not shown) cut into the upper surface 9 of the table 8. The cutting elements 14 cut a notch in the blister pack 1 as the tool tip 10 forms a line of weakening in the strip 1.

An effective line of weakness 6 need not be continuous. For example, one or more unweakened regions may be left in a line of weakness 6 in order to maintain the tensile strength of the strip 1 to, for example, facilitate handling during manufacture and indexing of the strip 1 in an inhalation device and to prevent accidental tearing of the strip 1. A line of weakness 6 may therefore comprise regions that have been weakened and regions that have not bees weakened or regions with differing levels of weakening.

It will be appreciated that more than one line of weakness 6 can be formed at one time by applying the weakening at a plurality of points simultaneously using for example, a tool with multiple blade members 9. Preferably, lines of weakness 6 are formed along a substantial portion or the whole of the strip 1 in a single operation. Alternatively they may be formed in a continuous process. Means for creating intermittent or continuous processes for high volume manufacture are well known in the field of blister processing machinery. Similarly, more than one strip can be processed at a time by processing strips side by side simultaneously.

FIG. 8 illustrates a further embodiment in which a laser 16 is used to form lines of weakness in the strip 1. According to this embodiment a laser beam 17 emitted by the laser 16 scans across the strip 1 in a predetermined pattern and locally modifies the polymer layer 2c by melting, ablation or a combination of the two, to form a line of weakness 6 without affecting the foil layer 2b. The laser 16 may be configured to ablate only a proportion of the thickness of the polymer layer 2c although controlling the accuracy of the depth of the ablation may be difficult in a continuous process where the foil layer 2b is subject to movement. Therefore, it is preferable if the laser 16 ablates the polymer layer 2c to its full depth. The laser 16 may ablate the ploymer layer 2c across only a portion of the width of the strip 1. For example in a dotted or dot matrix or dashed pattern such that along the line of weakness 6 there are alternate regions of ablated and unablated polymer, as shown in FIGS. 9A and 9B. In this way the strip can be made tearable without weakening it unduly.

The laser 16 may be a CO₂ laser or a YAG laser but is preferably a CO₂ laser. The type and power of the laser 16 is chosen to give effective ablation of the polymer layer 2c without damaging the aluminium foil layer 2b below. Forming a line of weakness with a laser can be combined with any of the aforementioned methods for initiating a tear including a nick, notch or kiss cut.

Advantageously for high volume production the process of making the blister strips 1 is continuous or comprises a combination of continuous and intermittent stations depending on the type of operation. For example, cold forming of a blister shape is often carried out by an intermittent process. In a continuous process, the laser 16 is programmed to scan across the foil layer 2b to form a line of weakness 6 and then index to the next position in synchronisation with the indexing of the strip 1 through the process. This may be achieved by scanning the laser beam 17 or by moving the strip 1. In an intermittent process the laser 16 may form a number of lines of weakness by scanning the beam 17 before the strip 1 is indexed along by a number of blisters 4.

It will be appreciated that all of the aforementioned embodiments address a method and apparatus for forming a region of weakness in a preformed blister strip. However, it is also envisaged that the formation of a region of weakness between blisters can be formed simultaneously with the manufacture of the blister i.e. at the same time as when the lid is sealed to the base.

FIG. 10A illustrates a portion of a sealing tool or plate 20 comprising a knurled or otherwise roughened patterned surface 21 that contacts the lidding material and performs the function of sealing it onto the base. The sealing tool 20 has an aperture 22 corresponding to a blister cavity so that the lidding material is unaffected by the tool 20 in the region of the lidding material that extends over the cavity. On either side of the aperture 22 is a raised area 23 for creating a tear propagation line of weakness in the strip on either side of a blister cavity. In a preferred embodiment, the raised area is substantially level with the tips or topmost surfaces of the knurling pattern which forms the seal. The raised areas 23 creates a region between the blisters which is both slightly thinned and stiffened as the laminate is squeezed against the sealing tool which is heated so as to cause the sealing lacquer to melt. This region is therefore slightly more brittle and easily torn once a tear is initiated.

It will be appreciated that the sealing tool may be applied to either side of the blister strip to seal the lidding material to the base. Furthermore, the lid and base material may be squeezed between two similar sealing tools.

FIG. 10B illustrates a modified version of a portion of the sealing tool shown in FIG. 10A. In this embodiment, the raised areas 25 take the form of enlarged regions at the edge of the strip between the apertures 22 to create tear initiation regions that facilitate the initiation of a tear. In a preferred embodiment, the raised region is substantially level with the tips or topmost surfaces of the kurled pattern which forms the seal. Alternatively, either of the raised areas shown in FIGS. 10A and 10B may be slightly proud of the knurled pattern by up to 0.2 mm. The knurled pattern is typically between 0.025 mm and 0.2 mm in depth and preferably between 0.04 mm and 0.1 mm.

FIG. 10C illustrates yet another modified version of a portion of the sealing tool shown in FIGS. 10A and 10B. In this version, the raised areas 23 as shown in FIG. 10A are combined with the raised areas 25 as shown in FIG. 10B. The combined raised region 23,25 is used to both initiate and propagate a tear. The tear initiation regions 25 may be slightly higher than the propagation regions 23 as the laminate needs to be weaker in the region of initiation.

Many modifications and variations of the invention falling within the terms of the following claims will be apparent to those skilled in the art and the foregoing description should be regarded as a description of the preferred embodiments of the invention only.

1. A blister pack comprising a plurality of spaced blister cavities each configured to receive and store an individual dose of medicament for inhalation by a user, wherein the pack includes a foil layer, an outer polymer layer and, a region of weakness formed by substantially removing or displacing a portion of the outer polymer layer from the foil layer between each blister cavity, or a number of blister cavities.

2. A blister pack according to claim 1, wherein the pack includes an inner polymer layer and the region of weakness is formed by substantially displacing the inner polymer layer, in addition to substantially removing or displacing the outer polymer layer, from the foil.

3. A blister pack according to claim 2, comprising a base portion in which the blister cavities are formed and a substantially planar lid portion sealing the blister cavities.

4. A blister pack according to claim 3, wherein the outer and inner polymer layers and the foil layer forms part of the base portion.

5. A blister pack according to claim 2, wherein the inner polymer layer has a thickness which is less than 60 μm.

6. A blister pack according to claim 5, wherein the inner polymer layer has a thickness of between 15 μm and 40 μm.

7. A blister pack according to claim 5, wherein the inner polymer layer has a thickness of 30 μm.

8. A blister pack according to claim 1 comprising an elongate strip of blisters.

9. A blister pack according to claim 8, wherein the region of weakness is provided between each blister of the strip.

10. A blister pack according to claim 1, wherein the or each region of weakness is discontinuous.

11. A blister pack according to claim 10, wherein the outer polymer layer is substantially removed or displaced from discrete, spaced apart regions extending along each region of weakness so that each region of weakness is formed from a series of weakened and non-weakened sections.

12. A blister pack according to claim 1, wherein the region of weakness is formed at an edge of a pack to to facilitate the initiation of a tear.

13. A blister pack according to claim 1, comprising a notch, nick or perforation in the edge of the pack to facilitate initiation of a tear.

14. A blister pack according to claim 1, wherein at least a portion of the region of weakness is a narrow strip or line.

15. A blister pack according to claim 14, wherein the region of weakness comprises a narrow strip or line and an enlarged region of weakness where the line meets an edge of the pack.

16. A blister pack according to claim 15, wherein the narrow strip or line extends across the pack between two enlarged regions of weakness where the ends of the line meet the edges of the pack.

17. A method of imparting a region of weakness in a blister pack between adjacent blister cavities of the pack which receive and store individual doses of medicament for inhalation by a user, the pack including a foil layer and an outer polymer layer, wherein the method includes the step of substantially removing or displacing a portion of the outer polymer layer from the foil layer to form said region of weakness.

18. A method according to claim 17, wherein the step of substantially removing or displacing a portion of the outer polymer layer from the foil layer to form said region of weakness includes the step of applying heat and pressure to the pack to soften or melt a portion of the outer polymer layer and compress and/or push said portion away from the foil in said region.

19. A method according to claim 18, including the step of cutting a portion of the pack in a region where a region of weakness meets an edge of the pack to form means for initiating a tear in the region of weakness.

20. A device for imparting a region of weakness to a blister pack between adjacent blister cavities of the pack which receive and store individual doses of medicament for inhalation by a user, the pack including a foil layer and an outer polymer layer, the device including means for heating and/or compressing a portion of the blister pack in a region where a region of weakness is to be formed so as to substantially remove or displace a portion of the outer polymer layer from the foil layer.

21. A device according to claim 20, wherein said means for heating and/or compressing a portion of the blister pack is a heated blade member.

22. A device according to claim 21, wherein the device includes a base member on which the blister pack is located and stop members depending from the heated blade member that engage the base member during formation of a region of weakness to maintain a predetermined distance between the heated blade member and the base member.

23. A device according to claim 21, wherein the device includes cutting members depending from the heated blade member to cut a portion of the pack in a region where a region of weakness meets the edge of the pack to form means for initiating a tear in a region of weakness.

24. A device according to claim 20, wherein the means for heating a portion of the blister pack in a region where a region of weakness is to be formed so as to substantially remove or displace a portion of the outer polymer layer from the foil layer is a laser.

25. A device according to claim 24, wherein the laser is configured to remove or displace a portion of the outer polymer layer from discrete separate regions along each region of weakness.

26. A sealing tool for manufacturing a blister pack, the pack comprising a base portion having a plurality of blister cavities therein and a lid portion for attachment to the base portion over the cavities to seal them once a dose has been located in each blister cavity, the pack including a foil layer and one or more polymer layers, wherein the sealing tool comprises a heated surface to compress and heat a blister base and lid positioned on the base to heat seal the lid to the base, wherein the sealing surface includes raised sections to substantially remove or displace a portion of the outer polymer layer from the foil layer to form a region of weakness as the lid is heat sealed to the base.

27. A sealing tool according to claim 26, wherein the heated surface is knurled and the raised regions are substantially level with the topmost surfaces of the knurling pattern.

28. A sealing tool according to claim 26, wherein the heated surface is knurled and the raised region is proud of the knurled pattern by up to 0.2 mm.

29. A sealing tool according to claim 23, wherein the raised region has a first section to form a region of weakness at the edge of a pack and a second section to form another region of weakness extending from the first section remote from the edges of the pack.

30. A sealing tool according to claim 29, wherein the height of the first section from the surface is greater than the height of the second section from the surface.

31. An inhalation device containing a blister pack according to claim 1.
